SEQUENCE LISTING (5 sequences)

SEQ ID NO: 1 - protein, 843 aa, Artificial Sequence
  ABCF1
MPKAPKQQPPEPEWIGDGESTSPSDKVVKKGKKDKKIKKTFFEELAVEDKAGEEEKVLKEKEQQQQQQQQQQKKKRDTRKGRRKKDVDDDGEEKELMERLKKLSVPTSDEEDEVPAPKPRGGKKTKGGNVFAALIQDQSEEEEEEEKHPPKPAKPEKNRINKAVSEEQQPALKGKKGKEEKSKGKAKPQNKFAALDNEEEDKEEEIIKEKEPPKQGKEKAKKAEQGSEEEGEGEEEEEEGGESKADDPYAHLSKKEKKKLKKQMEYERQVASLKAANAAENDFSVSQAEMSSRQAMENASDIKLEKFSISAHGKELFVNADLYIVAGRRYGLVGPNGKGKTTLLKHIANRALSIPPNIDVLLCEQEVVADETPAVQAVLRADTKRLKLLEEERRLQGQLEQGDDTAAERLEKVYEELRATGAAAAEAKARRILAGLGFDPEMQNRPTQKFSGGWRMRVSLARALFMEPTLLMLDEPTNHLDLNAVIWLNNYLQGWRKTLLIVSHDQGFLDDVCTDIIHLDAQRLHYYRGNYMTFKKMYQQKQKELLKQYEKQEKKLKELKAGGKSTKQAEKQTKEALTRKQQKCRRKNQDEESQEAPELLKRPKEYTVRFTFPDPPPLSPPVLGLHGVTFGYQGQKPLFKNLDFGIDMDSRICIVGPNGVGKSTLLLLLTGKLTPTHGEMRKNHRLKIGFFNQQYAEQLRMEETPTEYLQRGFNLPYQDARKCLGRFGLESHAHTIQICKLSGGQKARVVFAELACREPDVLILDEPTNNLDIESIDALGEAINEYKGAVIVVSHDARLITETNCQLWVVEEQSVSQIDGDFEDYKREVLEALGEVMVSRPRE

SEQ ID NO: 2 - DNA, 20 bp, Artificial Sequence
  GAPDH Forward Primer
tggatttggacgcattggtc

SEQ ID NO: 3 - DNA, 21 bp, Artificial Sequence
  GAPDH Reverse Primer
tttgcactggtacgtgttgat

SEQ ID NO: 4 - DNA, 21 bp, Artificial Sequence
  ABCF1 forward primer
agaaagcccgagttgtgtttg

SEQ ID NO: 5 - DNA, 21 bp, Artificial Sequence
  ABCF1 reverse primer
gcccccttgtagtcgttgatg

(19) United States
(12) Patent Application Publication
(10) Pub. No.: US 2023/0405026 A1
(43) Pub. Date: Dec. 21, 2023

(54) A Method of Treating Depression by Immune Modulation

(71) Applicants: Mynd Life Sciences Inc., Kelowna (CA); The University of British Columbia, Vancouver (CA)

(72) Inventor: Wilfred Jefferies, Surrey (CA)

(21) Appl. No.: 18/035,578

(22) PCT Filed: Nov. 5, 2021
(86) PCT No.: PCT/CA2021/051579

§ 371 Date: May 5, 2023

Related U.S. Application Data
(60) Provisional application No. 63/110,421, filed on Nov. 6, 2020.

(51) Int. Cl. A61K 31/675; A61K 31/4045; A61K 45/06; A61P 25/24; A61P 29/00

(52) U.S. Cl. CPC A61K 31/675; A61K 31/4045; A61K 45/06; A61P 25/24; A61P 29/00

(57) ABSTRACT

The present invention relates to methods of immune modulation. In particular, the present invention relates to regulation of neuroinflammation by modulation of ABCF1. Modulation of ABCF1 may be useful in the MDD.

### FIELD OF THE INVENTION

The present invention relates to methods of treating depression by immune modulation. In particular, the present invention relates to treatment of depression by immune modulation through modulation of ABCF1.

### BACKGROUND

Depression affects approximately 10% of humans globally, and the World Health Organization predicts it will become the third most prevalent disease in the world. New evidence indicates that some forms of anxiety and Major Depressive Disorder (MDD) are associated with chronic inflammation and autoimmune diseases including RA, and chronic inflammatory bowel diseases, such as Crohn's Disease (CD). MDD appears to be caused by both genetic and environmental factors, and its diagnosis and management is clinically challenging both because of its unpredictable presentation and response to treatment. Furthermore, MDD is associated with premature mortality from suicide. A traditional hypothesis is that patients living with MDD have a deficiency in brain monoamine neurotransmitters. However, some forms of MDD may be viewed as a psycho-neuroimmunological disorder, which may help to explain why therapies to reduce chronic inflammation also reduce depressive symptoms. Over the last decade, it has become increasingly apparent that several antidepressants possess anti-inflammatory properties. Mechanistically, antidepressants reduce levels of circulating pro-inflammatory cytokines (e.g. IL-1β, TNFα, and IL-6), and reciprocally increase levels of anti-inflammatory cytokines, including IL-10. Consequently, this altered cytokine milieu can modulate serotonergic signaling in neurons and thereby influence emotional and cognitive processing. Overall, these observations suggest that targeting cytokine regulatory pathways can be an effective and novel approach to treat depression.

Escitalopram, an antidepressant of the SSRI (selective serotonin receptor inhibitor) class, has been reported to influence anti-inflammatory pathways in patient populations and it was concluded that ABCF1, an E2 ubiquitin conjugating enzyme, which functions as a strong negative regulator of pro-inflammatory responses is Escitalopram's putative therapeutic target.

### SUMMARY OF THE INVENTION

An object of the present invention is to provide a method of treating depression by immune modulation.

In accordance with an aspect of the present invention, there is provided a method of inhibiting neuroinflammation in a patient in need thereof, said method comprising administering an agonist of ABCF1.

In accordance with an aspect of the present invention, there is provided a method inhibiting neuroinflammation in a patient in need thereof, said method comprising administering an ABCF1 protein or a polynucleotide encoding ABCF1.

In certain embodiments, inhibition of neuroinflammation treats or alleviates one or more symptoms of depression in said patient. In certain embodiments, the one or more symptoms are selected from the group consisting of trouble concentrating, remembering details, and making decisions; fatigue; feelings of guilt, worthlessness, and helplessness; pessimism and hopelessness; insomnia, early-morning wakefulness, or sleeping too much; irritability; restlessness; loss of interest in things once pleasurable, including sex; overeating, or appetite loss; aches, pains, headaches, or cramps that won't go away; digestive problems that don't get better, even with treatment; persistent sad, anxious, or “empty” feelings; suicidal thoughts or attempts and combinations thereof. In certain embodiments, the patient is a patient with an autoimmune disease, such as inflammatory bowel disease including Crohn's disease and ulcerative colitis, rheumatoid arthritis, or pancreatitis. In certain embodiments, the agonist treats or alleviates one or more symptoms of said autoimmune disease.

In accordance with an aspect of the present invention, there is provided a method of preventing and/or treating Major Depressive Disorder, said method comprising administering an agonist of ABCF1.

In accordance with an aspect of the present invention, there is provided a method of preventing and/or treating Major Depressive Disorder, said method comprising administering an ABCF1 protein or a polynucleotide encoding ABCF1.

In accordance with an aspect of the present invention, there is provided a method of treating an autoimmune disease and comorbid neuropsychiatric disorders, said method comprising administering an agonist of ABCF1.

In accordance with an aspect of the present invention, there is provided a method of treating an autoimmune disease and comorbid neuropsychiatric disorders, said method comprising administering an ABCF1 protein or a polynucleotide encoding ABCF1.

In accordance with an aspect of the present invention, there is provided a method of preventing and/or treating a depressive disorder associated with inflammation, said method comprising administering an agonist of ABCF1. In specific embodiments, the inflammation is neuroinflammation.

In certain embodiments, the agonist is psylocibin, psylocin or an analog or derivative thereof.

In certain embodiments, the agonist is psilocin, psylocibin, 4-Acetoxy-N, N-dimthyltryptamine, O-acetyl psilocin fumerate, and 4-acetoxyindole.

In certain embodiments, the agonist is a compound set forth in Table 1 or 2.

### BRIEF DESCRIPTION OF THE FIGURES

These and other features of the invention will become more apparent in the following detailed description in which reference is made to the appended drawings.

FIG. 1—illustrates that Escitalopram induces ABCF1 in a Macrophage cell line: RAW macrophages were plated at 1×105 cells/well and cultured for 2 days. The cells were incubated with 0.3 mM Escitalopram for 1 hour, and then harvested for total RNA, which was extracted for real time RT-PCR specific for ABCF1 and IL-4. CT values were normalized with CT value for the housekeeping gene from the DMSO control. The difference in the expression after drug treatment is consistent with polarization towards an M2-like phenotype (data were consistent in 3 separate experiments).

FIG. 2—illustrates the effect of psylocibin, psylocin and their analogs on ABCF1 transcription in a macrophage cell line. Briefly, the Macrophage cell line RAW264.7 (ATCC) were grown to 80% confluency in growth media (DMEM+ 10% FBS+ glutamine). Dilutions of psylocibin, psylocin and their analogs were made at desired final concentrations for a Dose response experiment. The concentrations' used for this experiment are: 10 nM, 100 nM, 500 nM for Psilocin, Psylocibin, 4-Acetoxy-N, N-dimthyltryptamine, O-Acetyl Psilocin Fumerate, and 4-acetoxyindole. The cells were incubated for 2 hours and then harvested for total RNA, which was extracted for real time RT-PCR specific for ABCF1. Untreated cells were used as negative control and Escitalopram at 0.3 mM was used as a positive control to activate ABCF1 expression for all the experiments. ES=escitalopram; PSYB=Psylocibin; PSIC=Psilocin; DMT=4-Acetoxy-N, N-dimthyltryptamine; APF=O-Acetyl Psilocin Fumerate, and AOI=4-acetoxyindole.

### DETAILED DESCRIPTION

The present invention is based on the discovery that some forms of anxiety and depression, including but not limited to Major Depressive Disorder (MDD) are associated with chronic inflammation and certain antidepressants possess anti-inflammatory properties. Accordingly, in certain embodiments, the present invention provides method of inhibiting inflammation, including but not limited to neuroinflammation. In specific embodiments, the present invention provides method of inhibiting inflammation, including but not limited to neuroinflammation, to treat neuropsychiatric disorders, including but not limited to Major Depressive Disorder (MDD), schizophrenia, anxiety, bipolar disorder, obsessive-compulsive disorder (OCD), posttraumatic stress disorder (PTSD), post-partum depression, autism spectrum disorder and other forms of clinical depression associated with inflammation.

Accordingly, in certain embodiments, the present invention provides methods of treating Major Depressive Disorder (MDD) or alleviating one or more symptoms of MDD. Symptoms of MDD include but are not limited to trouble concentrating, remembering details, and making decisions; fatigue; feelings of guilt, worthlessness, and helplessness; pessimism and hopelessness; insomnia, early-morning wakefulness, or sleeping too much; irritability; restlessness; loss of interest in things once pleasurable, including sex; overeating, or appetite loss; aches, pains, headaches, or cramps that won't go away; digestive problems that don't get better, even with treatment; persistent sad, anxious, or “empty” feelings; and suicidal thoughts or attempts.

ABCF1, an E2 ubiquitin conjugating enzyme, is a strong negative regulator of pro-inflammatory responses. Accordingly, neuroinflammation may be inhibited by upregulating the expression and/or activity of ABCF1. In certain embodiments, the present invention provides methods of inhibiting neuroinflammation by upregulating the expression and/or activity of ABCF1. In certain embodiments, the present invention provides methods of inhibiting neuroinflammation to treat neuropsychiatric disorders by upregulating the expression and/or activity of ABCF1. In specific embodiments, the present invention provides methods of treating Major Depressive Disorder (MDD) or alleviating one or more symptoms of MDD by upregulating the expression and/or activity of ABCF1.

It is known in the art that MDD is common in patients with autoimmune diseases, such as Rheumatoid Arthritis, inflammatory bowel disease, multiple sclerosis and pancreatitis.

It is also known that inhibition of inflammation and/or an immune response may also be useful in the prevention and/or treatment of such autoimmune diseases. Accordingly, methods which treat autoimmune diseases by inhibiting inflammation and/or an immune response may also be useful in the treatment of MDD in autoimmune patients having comorbid MDD.

Accordingly, in certain embodiments, the present invention provides methods of preventing and/or treating autoimmune and comorbid neuropsychiatric disorders. In certain embodiments, the present invention provides treatments for inflammatory autoimmune disease and neuropsychiatric disorders associated with neuroinflammation by immune modulation. In certain embodiments, the present invention provides combined treatments for inflammatory autoimmune disease and neuropsychiatric disorders by upregulating ABCF1 expression and/or activity. In specific embodiments, the present invention provides treatment for Rheumatoid Arthritis comorbid Major Depressive Disorder by upregulating ABCF1 expression and/or activity.

Non-limiting examples of methods to enhance expression and/or activity of ABCF1, include administration of the ABCF1, or active fragments thereof, administration of a nucleic acid or vector which encodes the ABCF1 or administration of one or more molecules which enhance expression of ABCF1.

The ABCF1 protein and nucleic acid sequences (genomic and cDNA) are known in the art. See for example GenBank Accession numbers AQY76226.1, AQY76225.1, KY500135.1 and KY500134.1. In certain embodiments, the ABCF1 comprises the sequence set forth below:

MPKAPKQQPP EPEWIGDGES TSPSDKVVKK GKKDKKIKKTFFEELAVEDKAGEEEKVLK
EKEQQQQQQQQQQKKKRDTRKGRRKKDVDDDGEEKELMERLKKLSVPTSDEEDEVPAP
KPRGGKKTKGGNVFAALIQDQSEEEEEEEKHPPKPAKPEKNRINKAVSEEQQPALKGKKG
KEEKSKGKAKPQNKFAALDNEEEDKEEEIIKEKEPPKQGKEKAKKAEQGSEEEGEGEEEEE
EGGESKADDPYAHLSKKEKKKLKKQMEYERQVASLKAANAAENDFSVSQAEMSSRQAME
NASDIKLEKFSISAHGKELFVNADLYIVAGRRYGLVGPNGKGKTTLLKHIANRALSIPPNIDVL
LCEQEVVADETPAVQAVLRADTKRLKLLEEERRLQGQLEQGDDTAAERLEKVYEELRATGA
AAAEAKARRILAGLGFDPEMQNRPTQKFSGGWRMRVSLARALFMEPTLLMLDEPTNHLDL
NAVIWLNNYLQGWRKTLLIVSHDQGFLDDVCTDIIHLDAQRLHYYRGNYMTFKKMYQQKQK
ELLKQYEKQEKKLKELKAGGKSTKQAEKQTKEALTRKQQKCRRKNQDEESQEAPELLKRP
KEYTVRFTFPDPPPLSPPVLGLHGVTFGYQGQKPLFKNLDFGIDMDSRICIVGPNGVGKSTL
LLLLTGKLTPTHGEMRKNHRLKIGFFNQQYAEQLRMEETPTEYLQRGFNLPYQDARKCLGR
FGLESHAHTIQICKLSGGQKARVVFAELACREPDVLILDEPTNNLDIESIDALGEAINEYKGAV
IVVSHDARLITETNCQLW VVEEQSVSQI DGDFEDYKRE VLEALGEVMVSRPRE

Appropriate vectors are known in the art and include but are not limited to adenoviral vectors.

Molecules known to enhance the ABCF1 pathway include but are is not limited to Escitalopram, an antidepressant of the SSRI. A number of psilocybins have immune modulatory activities and may enhance the ABCF1 pathway. Accordingly, in certain embodiments, the methods comprise administration of psilocybins, analogs and derivatives thereof and/or antidepressant drugs to modulate the ABCF1 pathway.

In certain embodiments, the molecule is escitalopram or molecules structurally similar to escitalopram.

In certain embodiments, the molecule is selected from any one of the following set forth in Table 1:

Escitalopram oxalate
Escitalopram hydrobromide
Escitalopram
Escitalopram(1+)
Citalopram
1-[4-Bromo-2-(hydroxymethyl)phenyl]-4-(dimethylamino)-1-(4-fluorophenyl)butan-1-ol
(S)-Citalopram N-Oxide
Desmethyl Citalopram Hydrobromide
2,3-Bis[(4-methylbenzoyl)oxy]butanedioic acid; 4-[4-(dimethylamino)-1-(4-fluorophenyl)-1-
hydroxybutyl]-3-(hydroxymethyl)benzonitrile
4-[4-(Dimethylamino)-1-(4-fluorophenyl)-1-hydroxybutyl]-3-
(hydroxymethyl)benzonitrile; methane
3-[(1S)-1-(4-Fluorophenyl)-5-isocyano-3H-2-benzofuran-1-yl]-N-methyl-N-
(trideuteriomethyl)propan-1-amine; oxalic acid
1-(4-Fluorophenyl)-1-(3-dimethylaminopropyl)-5-chlorophthalane
4-[(Z)-4-(Dimethylamino)-1-(4-fluorophenyl)but-1-enyl]-3-(hydroxymethyl)benzonitrile
1,3-Dihydro-1-oxoisobenzofuran-5-carboxamide
1-(3-Dimethylaminopropyl)-1-(4-fluorophenyl)-1,3-dihydroisobenzofuran-5-carboxamide
Citalopram Oxalate
desmethylcitalopram (DCT)
didesmethylcitalopram (DDCT)
(RS)-1-[3-(Dimethylamino)propyl]-1-(4-fluorophenyl)-1,3-dihydroisobenzofuran-5-carbonitrile
citalopram propionaldehyde
citalopram propionic acid
(RS)-1-[3-(methylethylamino)propyl]-1-(4-fluorophenyl)-1,3-dihydroisobenzofuran-5-
carbonitrile
(RS)-1-[3-(Dieethylamino)propyl]-1-(4-fluorophenyl)-1,3-dihydroisobenzofuran-5-carbonitrile

In certain embodiments, the molecule is a derivative of escitalopram. In certain embodiments, the middle carbon atom middle carbon atom of the propyl linkage from the N atom to the chiral centre comprises a methyl or ethyl substitution. In certain embodiments, the 4-fluorophenyl group in the 2 and/or 6 positions (meta to the F) is substituted. For example, the 2,4-difluorophenyl substructure is known in 1-(2,4-difluorophenyl)methanamine and the 2,4,6-trifluorophenyl substructure is known in 2,4,6-Trifluorophenyl)methanamine—both of which are available as reagents. In certain embodiments, the molecule is a deuterated escitalopram.

In certain embodiments, the molecule is a psilocybin, analog thereof, derivative thereof or a psilocybin-like molecule.

In certain embodiments, the psilocybin derivatives share a tryptamine core attached to an ethyl amino group, as illustrated below. The groups designated with an R can be varied as needed to define specific molecules in this chemical family.

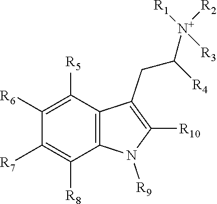

In specific embodiments, the molecule is any one of the following set forth in table 2:

Psilocybin
Psilocin
4-Hydroxytryptamine
O-Methylpsilocin
4-Hydroxy-N,N diethyltryptamine
3-(2-Pyrrolidin-1-yl-ethyl)-1H-indol-4-ol
Indol-6-OL, 3-(2-(dimethylamino)ethyl)-
4-Hydroxy-N-methyltryptamine 4-phosphate
4-Hydoxytryptamine 4-phosphate
3-(2-Dimethylamino-propyl)-1H-indol-4-ol
4-Hydroxy-N,N-diisopropyltryptamine
4-Hydroxy-N-methyl-N-ethyltryptamine
4-Hydroxy-N-methyl-N-isopropyltryptamine
[3-[2-(Dimethylamino)ethyl]-1-methylindol-4-yl] dihydrogen phosphate
Ethocybin
3-(3-Dimethylamino-propyl)-1H-indol-4-ol
3-Dimethylaminomethyl-1H-indol-4-ol
[1-Butyl-3-[2-(dimethylamino)ethyl]indol-4-yl] dihydrogen phosphate
2-(4-Hydroxy-1H-indol-3-yl)ethanaminium
3-[2-(Dimethylazaniumyl)ethyl]-1H-indol-4-yl phosphate
3-[2-(4-Methyl-piperazin-1-yl)-ethyl]-1H-indol-4-ol
Meprocin
3-[2-(Methylamino)ethyl]-1H-indol-4-ol
3-(2-Dimethylamino-ethyl)-6-fluoro-1H-indol-4-ol
3-(1-Methyl-pyrrolidin-3-yl)-1H-indol-4-ol
Norbaeocystin
Baeocystin
[3-[2-(Trimethylazaniumyl)ethyl]-1H-indol-4-yl] phosphate
N,N-Diallyl-4-hydroxytryptamine
Aeruginascin
n,n-Diisopropyl-4-hydroxytryptamine hydrochloride
Dipropyl-4-hydroxytryptamine
3-[(2R)-2-Aminopropyl]-1H-indol-4-ol
3-[(2S)-2-Aminopropyl]-1H-indol-4-ol
(R)-3-((1-Methylpyrrolidin-2-YL)methyl)-1H-indol-4-OL
3-Methyl-1H-indol-4-ol
O-Benzyl Psilocybin
3-(2-Methylbut-3-en-2-yl)-1H-indol-4-ol
1H-Indol-4-olate
2-(4-Dibenzylphosphoryloxy-1H-indol-3-yl)-N,N-dimethylethanamine
4-Dibenzylphosphoryloxy-3-(2-piperidin-1-ylethyl)-1H-indole
1H-Indol-4-yl phosphate
Tris[[3-[2-(dimethylamino)ethyl]-1H-indol-4-yl]oxy]-hydroxyphosphanium
3-(2-Azidoethyl)-1H-indol-4-ol
3-(4-Azidooxy-1H-indol-3-yl)-N,N-dimethylpropan-1-amine
4-[Azido(methyl)phosphoryl]oxy-1H-indole
[3-[2-(Dimethylamino)ethyl]-1H-indol-4-yl] phosphate
[3-[2-[Benzyl(dimethyl)azaniumyl]ethyl]-1H-indol-4-yl] phosphate
4-Azidooxy-3-methyl-1H-indole
1H-Indol-4-yl sulfate
2-[1H-Indol-3-yloxy(methoxy)phosphoryl]oxyethyl-trimethylazanium
[3-[2-(Dimethylamino)ethyl]-1H-indol-4-yl]oxy-methylphosphinic acid
2-(4-Dihydroxyphosphanyloxy-1H-indol-3-yl)ethyl-trimethylazanium
[3-(3-Methylbutyl)-1H-indol-4-yl] phosphono hydrogen phosphate
2-(4-Dimethylphosphoryloxy-1H-indol-3-yl)-N,N-dimethylethanamine
2-[4-[Hydroxy(phenylmethoxy)phosphoryl]oxy-1H-indol-3-yl]ethyl-dimethylazanium
3-[2-[Amino(methyl)amino]ethyl]-1H-indol-4-ol
Benzyl-[2-[4-[hydroxy(methyl)phosphoryl]oxy-1H-indol-3-yl]ethyl]-dimethylazanium
3-[2-[Iodo(methyl)amino]ethyl]-1H-indol-4-ol
Potassium; 3-(2-aminoethyl)-1H-indol-4-olate
N-(1H-Indol-4-yloxy)-2-methylpropan-1-amine; methanol
O-[3-(2-Aminoethyl)-1H-indol-4-yl]hydroxylamine; fluoromethane
N,N-Dimethyl-2-(4-sulfanyloxy-1H-indol-3-yl)ethanamine; ethane
Ethane; methane; O-[3-[2-(methylamino)ethyl]-1H-indol-4-yl]hydroxylamine
3-Ethyl-4-sulfanyloxy-1H-indole; methane
(3-Heptan-3-yl-1H-indol-4-yl) hypofluorite; methane
Ethane; N-methyl-2-(4-sulfanyloxy-1H-indol-3-yl)ethanamine
Fluoromethane; (3-heptan-3-yl-1H-indol-4-yl) hypofluorite
Fluoromethane; O-[3-[2-(methylamino)ethyl]-1H-indol-4-yl]hydroxylamine
Methane; (3-pentan-3-yl-1H-indol-4-yl) hypofluorite; propane
Fluoromethane; octane; O-(3-propyl-1H-indol-4-yl)hydroxylamine
[3-(3-Ethylheptyl)-1H-indol-4-yl] hypofluorite; methane
3-(2-Aminoethyl)-1H-indol-4-ol; ethane
3-[2-(Dimethylamino)ethyl]-1H-indol-4-ol; ethane
1H-Indol-4-yloxyboronic acid
4-Methylsulfanyloxy-1H-indole
2-(5-Phosphorosooxy-1H-indol-3-yl)ethanamine
[3-[2-(Dimethylamino)ethyl]-1H-indol-4-yl] phosphono hydrogen phosphate
Benzyl-dimethyl-[2-(4-phosphonooxy-1H-indol-3-yl)ethyl]azanium
[3-[2-(Dimethylazaniumyl)ethyl]-1H-indol-4-yl] hydrogen phosphate
[3-[2-(Dimethylamino)ethyl]-1H-indol-4-yl] dihydrogen phosphate; hydrate
Dibenzyl [3-[2-(dimethylamino)ethyl]-1H-indol-4-yl] phosphate
3-[2-[Ethyl(propyl)amino]ethyl]-1H-indol-4-ol
[3-(1-Aminoethyl)-1H-indol-4-yl] dihydrogen phosphate
[3-[2-(Dimethylamino)ethyl]-1H-indol-4-yl] hydrogen phosphate
[1-(Aminomethyl)-3-[2-(methylamino)ethyl]indol-4-yl] hypoiodite
2-[4-(Methoxymethylphosphanyloxy)-1H-indol-3-yl]-N,N-dimethylethanamine
(2S)-2-[[[3-[2-(Dimethylamino)ethyl]-1H-indol-4-yl]oxy-(methoxymethyl)phosphoryl]amino]-2,3-
dimethylbutanoic acid
Dibromo 1H-indol-4-yl phosphate
3-[2-(Diethylamino)ethyl]-1H-indol-6-ol
3-[2-(Ethylamino)ethyl]-1H-indol-4-ol
1H-Indol-5-yl dihydrogen phosphate
3-[2-(Methylamino)ethyl]-1-propan-2-ylindol-4-ol
1H-Indol-4-yl hypofluorite
[4-(1-Ethyl-3,6-dihydro-2H-pyridin-5-yl)-1H-indol-3-yl] dihydrogen phosphate
3-[2-(Dimethylamino)ethyl]-5-fluoro-1H-indol-4-ol
3-[2-(Dimethylamino)ethyl]-1H-indol-4-ol; [3-[2-(dimethylamino)ethyl]-1H-indol-4-yl] dihydrogen
phosphate
4-Phosphorosooxy-1H-indole
1H-Indol-4-yl dihydrogen phosphate
O-[3-[2-(Methylamino)ethyl]-1H-indol-6-yl]hydroxylamine
3-(2-Methyl-1,2,3,6-tetrahydropyridin-4-yl)-1H-indol-4-ol
CID 67465946
1H-Indol-4-yloxy(oxo)borane
[3-[2-(Trimethylazaniumyl)ethyl]-1H-indol-4-yl] hydrogen phosphate
[3-[2-(Dimethylamino)ethyl]-7-fluoro-1-methylindol-4-yl] dihydrogen phosphate
[3-[2-(Dimethylamino)ethyl]-7-fluoro-1-propylindol-4-yl] dihydrogen phosphate
1-Ethyl-3-[2-(methylamino)ethyl]indol-4-ol; tungsten
CID 58110672
2-(4-Phosphorosooxy-1H-indol-3-yl)ethanamine
N,N-Dimethyl-2-(4-phosphorosooxy-1H-indol-3-yl)ethanamine
4-Hydroperoxy-1H-indole
1H-Indol-4-yl hypochlorite
3-Butyl-1H-indol-4-ol
1H-Indol-4-yl nitrate
O-(1H-Indol-4-yl)hydroxylamine
1H-Indol-4-yl hypobromite
1H-Indol-4-yl hypoiodite
3-[(1-Methylpyrrolidin-2-yl)methyl]-1H-indol-4-ol
4-Methylperoxy-1H-indole
N,N-Diethyl-2-(4-methoxy-1H-indol-3-yl)ethanamine
3-[[2-(Dimethylamino)phenyl]methyl]-1H-indol-4-ol
3-(2-Piperidinoethyl)-1H-indole-4-ol
3-[2-(Dibutylamino)ethyl]-1H-indol-4-ol
4-HO-Dsbt
3-(Aminomethyl)-1H-indol-4-ol
1H-Indol-4-ol, 3-ethyl-
4-Hydroxy-3-(1-methyl-1,2,3,6-tetrahydropyridin-4-yl)-1H-indole
4-Hydroxy-3-(1-benzyl-1,2,3,6-tetrahydropyridin-4-yl)-1H-indole
4-Hydroxy-3-(1,2,3,6-tetrahydropyridin-4-yl)-1H-indole
1-(4-Hydroxy-1H-indol-3-yl)ethyl-trimethylazanium
2-(4-Hydroxy-1H-indol-3-yl)ethyl-trimethylazanium
2-(4-Hydroxy-1H-indol-3-yl)ethyl-trimethylazanium; methyl sulfate
(4-Hydroxy-1H-indol-3-yl)methyl-trimethylazanium
(4-Hydroxy-1H-indol-3-yl)methyl-trimethylazanium; methyl sulfate
Benzyl [3-[2-[benzyl(dimethyl)azaniumyl]ethyl]-1H-indol-4-yl] phosphate
2-[Hydroxy(1H-indol-3-yloxy)phosphoryl]oxyethyl-trimethylazanium
1H-Indol-3-yl (2-(trimethylammonio)ethyl) phosphate
3-[2-[Methyl((111C)methyl)amino]ethyl]-1H-indol-4-ol
3-[[(2S)-1-Methyl-2-pyrrolidinyl]methyl]-1H-indole-4-ol
Benzyl-[2-[4-[hydroxy(phenylmethoxy)phosphoryl]oxy-1H-indol-3-yl]ethyl]-dimethylazanium
1H-Indol-4-ol, 3-(2-aminopropyl)-
3-[2-[Bis(2-methylpropyl)amino]ethyl]-1H-indol-4-ol
3-[2-[Butyl(methyl)amino]ethyl]-1H-indol-4-ol
3-[2-[Methyl(2-methylpropyl)amino]ethyl]-1H-indol-4-ol
3-[2-[Butan-2-yl(methyl)amino]ethyl]-1H-indol-4-ol
3-[2-[Tert-butyl(methyl)amino]ethyl]-1H-indol-4-ol
3-[2-[Cyclopentyl(methyl)amino]ethyl]-1H-indol-4-ol
3-[2-(2,6-Dimethylpiperidin-1-yl)ethyl]-1H-indol-4-ol
3-[2-(Dimethylamino)ethyl]-1H-indol-4-ol; phosphoric acid
2-(4-Hydroxy-1H-indol-3-yl)ethyl-dimethylazanium
4-Hydroxy-N-isopropyl-N-methyltryptamine-d4
1-Ethyl-3-[2-(methylamino)ethyl]indol-4-ol
3-[2-(N,2-Dimethylanilino)ethyl]-1H-indol-4-ol
Benzyl [3-[1,1,2,2-tetradeuterio-2-(dimethylamino)ethyl]-1H-indol-4-yl] hydrogen phosphate
N,N-Dimethyl-2-(4-trimethylsilyloxy-1H-indol-3-yl)ethanamine
3-[1,1,2,2-Tetradeuterio-2-[ethyl(methyl)amino]ethyl]-1H-indol-4-ol
3-[1,1,2,2-Tetradeuterio-2-(diethylamino)ethyl]-1H-indol-4-ol
3-[1,1,2,2-Tetradeuterio-2-(dimethylamino)ethyl]-1H-indol-4-ol
[3-[1,1,2,2-Tetradeuterio-2-(dimethylamino)ethyl]-1H-indol-4-yl] dihydrogen phosphate
1H-Indol-4-ol, 3-(3-aminopropyl)-
3-[2-(Methylamino)propan-2-yl]-1H-indol-4-ol
3-Methyl-5-[2-(methylamino)ethyl]-1H-indol-4-ol
5-(3-Aminopropyl)-3-methyl-1H-indol-4-ol
3-(2-Aminoethyl)-1-methylindol-4-ol
1-Methyl-3-[2-(methylamino)ethyl]indol-4-ol
3-(3-Aminopropyl)-1-methylindol-4-ol
3-(1-Aminopropan-2-yl)-1-methylindol-4-ol
3-(2-Aminopropyl)-1-methylindol-4-ol
3-(Methylaminomethyl)-1H-indol-4-ol
3-[2-(Methylamino)propyl]-1H-indol-4-ol
3-(2-Aminoethyl)-1-propan-2-ylindol-4-ol
1-Methyl-3-[2-(methylamino)propyl]indol-4-ol
3-(3-Aminopropyl)-1-propan-2-ylindol-4-ol
3-(2-Aminopropyl)-1-propan-2-ylindol-4-ol
3-(4-Piperidinyl)-1H-Indol-4-ol
1-Methyl-3-(1,2,3,6-tetrahydropyridin-4-yl)indol-4-ol
1-(3-Aminopropyl)-3-methylindol-4-ol
3-(2-Aminoethyl)-5-ethyl-1H-indol-4-ol
3-(Fluoromethyl)-1H-indol-4-ol
3-(1-Fluoroethyl)-1H-indol-4-ol
3-(2-Fluoropropan-2-yl)-1H-indol-4-ol
3-(Pyrrolidin-1-ylmethyl)-1H-indol-4-ol
3-(Piperidin-1-ylmethyl)-1H-indol-4-ol
3-(2-Aminoethyl)-1-ethylindol-4-ol
3-(2-Aminoethyl)-1-propylindol-4-ol
3-(2-Aminoethyl)-1-prop-2-enylindol-4-ol
[3-[2-(Dimethylamino)ethyl]-1-trimethylsilylindol-4-yl] bis(trimethylsilyl) phosphate
3-[2-[Hydroxy(methyl)amino]propan-2-yl]-1H-indol-4-ol
3-[[(3R)-1-Methyl-3-pyrrolidinyl]methyl]-1H-indole-4-ol
3-[[(3S)-1-Methyl-3-pyrrolidinyl]methyl]-1H-indole-4-ol
3-Propan-2-yl-1H-indol-4-ol
3-[2-[Bis(trideuteriomethyl)amino]-1,1,2,2-tetradeuterioethyl]-1H-indol-4-ol
3-(1-Aminopropan-2-yl)-1-ethylindol-4-ol
3-(2-Aminopropyl)-1-ethylindol-4-ol
3-(3-Aminopropyl)-1-ethylindol-4-ol
3-[(Cyclobutylamino)methyl]-1H-indol-4-ol
3-[(Cyclopentylamino)methyl]-1H-indol-4-ol
3-(Azepan-1-ylmethyl)-1H-indol-4-ol
3-[(4-Methylpiperazin-1-yl)methyl]-1H-indol-4-ol
3-(Ethylaminomethyl)-1H-indol-4-ol
3-[(Propan-2-ylamino)methyl]-1H-indol-4-ol
3-(Ethylaminomethyl)-1-methylindol-4-ol
1-Methyl-3-[(propan-2-ylamino)methyl]indol-4-ol
1-Ethyl-3-(methylaminomethyl)indol-4-ol
1-Ethyl-3-(ethylaminomethyl)indol-4-ol
1-Ethyl-3-[(propan-2-ylamino)methyl]indol-4-ol
3-(Ethylaminomethyl)-1-propan-2-ylindol-4-ol
1-Propan-2-yl-3-[(propan-2-ylamino)methyl]indol-4-ol
3-(2-Hydroxypropan-2-yl)-1H-indol-4-ol
3-(2-Hydroxy-2-methylpropyl)-1H-indol-4-ol
3-(Aminooxymethyl)-1H-indol-4-ol
1H-Indol-4-ol, 3-(1-aminoethyl)-
3-[1-(Methylamino)ethyl]-1H-indol-4-ol
1-Methyl-3-[1-(methylamino)ethyl]indol-4-ol
3-[3-(Methylamino)propyl]-1H-indol-4-ol
1-Methyl-3-[3-(methylamino)propyl]indol-4-ol
1-Ethyl-3-[1-(methylamino)ethyl]indol-4-ol
3-[1-(Methylamino)ethyl]-1-propan-2-ylindol-4-ol
1-Ethyl-3-[2-(methylamino)propyl]indol-4-ol
3-[2-(Methylamino)propyl]-1-propan-2-ylindol-4-ol
1-Ethyl-3-[3-(methylamino)propyl]indol-4-ol
3-[3-(Methylamino)propyl]-1-propan-2-ylindol-4-ol
5-(4-Aminobutyl)-3-methyl-1H-indol-4-ol
1H-Indol-4-ol, 3-propyl-
3-[2-(Dimethylamino)-1-hydroxyethyl]-1H-indol-4-ol
3-[(3S)-1-Methylpyrrolidin-3-yl]-1H-indol-4-ol
3-[(3R)-1-Methylpyrrolidin-3-yl]-1H-indol-4-ol
3-[1-(Dimethylamino)propan-2-yl]-1H-indol-4-ol
3-(2-Aminobutyl)-1H-indol-4-ol
3-(1-Amino-3-methylbutyl)-1H-indol-4-ol
3-[(1S)-1-Amino-3-methylbutyl]-1H-indol-4-ol
3-[(1R)-1-Amino-3-methylbutyl]-1H-indol-4-ol
3-[1-[Hydroxy(methyl)amino]ethyl]-1H-indol-4-ol
3-(N-Hydroxy-C-methylcarbonimidoyl)-1H-indol-4-ol
[3-[2-(Dimethylazaniumyl)ethyl]-1H-indol-4-yl] hydrogen phosphate; methanol
[3-(3-Ethylheptyl)-1H-indol-4-yl] hypofluorite
O-(3-Propyl-1H-indol-4-yl)hydroxylamine
(3-Pentan-3-yl-1H-indol-4-yl) hypofluorite
O-[3-[2-(Methylamino)ethyl]-1H-indol-4-yl]hydroxylamine
(3-Heptan-3-yl-1H-indol-4-yl) hypofluorite
N-Methyl-2-(4-sulfanyloxy-1H-indol-3-yl)ethanamine
3-Ethyl-4-sulfanyloxy-1H-indole
N,N-Dimethyl-2-(4-sulfanyloxy-1H-indol-3-yl)ethanamine
O-[3-(2-Aminoethyl)-1H-indol-4-yl]hydroxylamine
N-(1H-Indol-4-yloxy)-2-methylpropan-1-amine
1H-Indol-4-yl hydrogen sulfate

In certain embodiments, the molecule is selected from the group consisting of Psilocybin [3-(2-Dimethylaminoethyl)-1H-indol-4-yl] dihydrogen phosphate), Psilocybin (zwitterion form), Psilocin (4-hydroxy-N,N-dimethyltryptamine), Serotonin (5-Hydroxytryptamine), DMT (N,N-Dimethyltryptamine), Lysergic acid diethylamide (LSD, (6aR,9R)—N,N-diethyl-7-methyl-4,6,6a,7,8,9-hexahydroindolo[4,3-fg]quinoline-9-carboxamide, psilocin iminoquinone, psilocin o-quinone, Trimethylglycine (TMG), 0-acetyl psilocin fumarate, Phenyl hydrogen sulfate and indoxyl sulfate.

In certain embodiments, the molecule is selected from the group consisting of Psilocin, 4-Acetoxy-N-isopropyl-N-methyltryptamine, 4-Acetoxyindole, O-Acetyl Psilocin Fumarate, Psilocybin, 4-Acetoxy-N,N-dimethyltryptamine, 4-Acetoxy-N-isopropyl-N-methyltryptamine, 4-Acetoxy-N-ethyl-N-methyltryptamine, 4-Acetoxyindole, O-Acetyl Psilocin Fumarate, 4-AcO-DET Fumarate, 4-AcO-MET Fumarate, 4-AcO-DET, Baeocystin, O-Benzyl Psilocin, 3-(2-(Dimethylamino)ethyl)-5,6,7,7a-tetrahydro-1H-indol-4(3aH)-one, 4-Hydroxy-N-isopropyl-N-methyltryptamine, 4-Hydroxy McPT Hydrochloride, O-Methylpsilocin, 1-Methylpsilocin, 4-Methoxy MiPT Hydrochloride, (-)-Normacromerine, Psilocin O-Glucuronide.

The agonists of ABCF1 may be used alone or in combination with other therapeutic agents.

Psilocybin is present in mushrooms from the following genera: Agrocybe, Amanita, Conocybe, Galerina, Gymnopilus, Hypholoma, Inocybe, Panaeolus, Psilocybe, Pholiotina, Pluteus, and Weraroa. Exemplary Psilocybe include P. cubensis and P. subcubensis. P. semilanceata. Accordingly, the agonists for use in the methods of the present invention may be in the form of natural products or extracts from natural products. Methods of extracting psilocybin from mushrooms or producing psilocybin are known in the art. See U.S. Pat. No. 3,183,172 describing obtaining psilocybin and psilocin from fungal material and U.S. Pat. No. 10,519,175 directed to preparations of psylocybin and polymorphs of psylocybin.

In certain embodiments, there is provided bioassay screens which utilize ABCF1 to identify new drugs for treatment of MDD. In certain embodiments, there is provided bioassay screens which utilize ABCF1 to identify new drugs for treating autoimmune and comorbid neuropsychiatric disorders. For example, the screens may be used to identify drugs that modulate an immune response.

In certain embodiments, there is provided methods to determine ABCF1 expression. Such methods may be used to identify agents that modulate ABCF1 expression and therefore may be useful in the identification of drugs. In specific embodiments, a reporter gene is placed under the control of the ABCF1 promoter and the reporter gene product is measured (either qualitatively or quantitatively). Cells, including but not limited macrophages such as RAW 264.7 cell line, comprising the ABCF1 promoter reporter gene product may be used in assays to identify agents that modulate ABCF1 expression.

To gain a better understanding of the invention described herein, the following examples are set forth. It will be understood that these examples are intended to describe illustrative embodiments of the invention and are not intended to limit the scope of the invention in any way.

### Example 1

Major depressive disorder (MDD), often referred to as “depression’, affects psychosocial functioning and diminishes the quality of life¹. It affects over 300 million people worldwide² and is associated with ˜800,000 suicide deaths annually³. The World Health Organization states that MDD will become the third most prevalent disease in the World by 2030⁴. It occurs in higher prevalence in women than in men, but the aetiology of depression remains poorly understood. It appears to be caused by both genetic and environmental factors, however, its diagnosis and management are clinically challenging because of unpredictable presentation and response to treatment⁴. Furthermore, depression remains associated with premature mortality from suicide and other illnesses⁵. A traditional hypothesis is that those living with depression have a deficiency in monoamine neurotransmitters such as serotonin and norepinephrine in the brain, however, evidence now shows that some forms of depression are associated with ongoing forms of low-grade inflammation⁶.

Subsets of depression patients have an impaired peripheral immune system, increased levels of proinflammatory cytokines that can affect neurotransmitter metabolism, neuroendocrine function and regional brain activity⁷. Patients given proinflammatory cytokines, such as IL-1b, experience more symptoms of anxiety and depression than untreated patients⁷, and patients experiencing bacterial and viral infections often experience symptoms associated with depression (i.e. disrupted sleep, fatigue, depressed moods, impaired concentration)⁸.

Studies link MDD to higher levels of inflammatory markers compared to those who are not clinically depressed. A study of >14,000 patients showed those with depression had 46% higher levels of C-reactive protein (CRP), an inflammation marker, in their blood 8 The immune balance between Th1/Th2 and Th17/Treg correlate with MDD¹⁷. Depressed subjects have an increase in peripheral Th17 cell number and a decrease in T-reg cell number resulting in imbalance of Th17/Treg ratio compared to healthy controls¹⁸. Furthermore, studies show that pregnant patients with MDD have elevated inflammatory responses^{19,20} and higher levels of circulating steroids compared to healthy pregnant women 2. Specifically women exhibiting severe depression (SD) and severe anxiety (SA) during pregnancy exhibit high levels of Th1-(IL-6, TNF-α, IL-2, IFN-γ), Th17-(IL-17A, IL-22), and Th2- (IL-9, IL-10, and IL-13) related cytokines. The SA group alone showed higher concentrations of Th1-(IL-6, TNF-α, IL-2, IFN-γ) and Th2-(IL-4, and IL-10) cytokines versus the controls¹⁷.

Moreover, the immune balance between M1/M2 macrophages has previously been proposed as a target of therapy for MDD. Studies on humans and animals have documented that chronic activation of M1 microglial cells²³⁻²⁵ may trigger mood disorders²⁶ through the release of a variety of chemokines, eicosanoids, free radicals, neurotoxins, pro-inflammatory cytokines, and nitric oxide²³, thereby potentiating neuronal dysfunction 27. Various bacterial and viral infections including influenza virus, Herpes viruses, and HIV induce the secretion of proinflammatory cytokines and induce microglial activation that is associated with depression symptoms²³⁻³². Experimental induction in humans with immune activators that activate microglia such as endotoxin (LPS) a key driver of SIRS or gram-negative bacteria such as Salmonella typhimurium induces depressive symptoms, where the severity is correlated with elevated blood levels of inflammatory cytokines 33-35. In animal models, LPS administration induces microglial activation together with depression symptoms in rodents that is halted with selective serotonin reuptake inhibitors (SSRIs) or tricyclic antidepressants (TCAs)^{36,37}. In fact, many observations support the involvement of microglia in LPS-induced depression: (i) LPS-induced depression symptoms can be reduced by treatment with the microglial inhibitor minocycline 38; (ii) activation of the enzyme indoleamine 2,3-dioxygenase (IDO) in microglia is essential for the development of depression symptoms and microglial activation induced by LPS³⁸⁻⁴¹; and (iii) mice with microglial hyper-reactivity by traumatic brain injury 42, or induced by a microglia-specific mutation in the fractalkine receptor 43 exhibit heightened LPS-induced depression symptoms. In contrast, mice deficient in NLRP3 inflammasome signalling resulting in induction of pro-inflammatory cytokine secretion have attenuated depression in response to LPS⁴⁴.

ABCF1 is a missing link in inflammatory disease and depression. ABCF1 is a protein within the ABC (ATP-binding cassette) gene, family. Unlike other ABC family members, ABCF1 lacks the transmembrane domain and does not appear to function as a transporter. The ABCF1 gene is located in the class I region of the major histocompatibility complex locus on chromosome 6 in humans and on chromosome 17 in mice. Previous studies have shown that ABCF1 participates in translation initiation through its association with eIF2 and ribosomes 45-49. ABCF1 is known to be located in the cytoplasm and nucleoplasm, but not in the nucleolus 47. Gene expression of ABCF1 has been shown to be elevated substantially in human synoviocytes isolated from the inflamed joints of rheumatoid arthritis patients, and this increases further when stimulated with TNF-α⁵⁰. Also, the ABCF1 locus is linked to increased susceptibility to autoimmune pancreatitis in the Japanese population⁵¹ and, importantly, ABCF1 has been associated with susceptibility to rheumatoid arthritis in European and Asian populations⁵². Immunological studies in mouse embryonic fibroblasts have shown that ABCF1 associates with dsDNA and DNA sensing components HMGB1 and IF1204, and further interacts with SET complex members (SET, ANP32A and HMGB2) to facilitate cytosolic DNA sensing mechanisms.

ABCF1 acts as a ubiquitin-switch that regulates inflammatory pathways. Although ABCF1 (+/) mice appear normal under specific pathogen-free conditions, we recently discovered that ABCF1 acts as a molecular switch between inflammatory pathways downstream of TLRs 53. In the Immunity paper, “The ATP-Binding Cassette Gene ABCF1 Functions as an E2 Ubiquitin-Conjugating Enzyme Controlling Macrophage Polarization to Dampen Lethal Septic Shock” (2019) 53, sepsis was studied, where little was known regarding the molecular switches and pathways that regulate this disease. It was discovered that ABCF1 possesses an E2 ubiquitin enzyme activity, through which it controls the LPS-Toll-like Receptor-4 (TLR4)-mediated gram-negative insult by targeting key proteins for K63-polyubiquitination. K63-ubiquitination by ABCF1 shifts the inflammatory profile from an early phase MyD88-dependent to a late phase TRIF-dependent signalling pathway, thereby regulating TLR4 endocytosis and modulating macrophage polarization from M1 to M2 phase. Physiologically, ABCF1 regulates the shift from the inflammatory phase of sepsis to the endotoxin tolerance phase and modulates cytokine storm and interferon-β-dependent production by the immunotherapeutic mediator, SIRT1. Consequently, ABCF1 controls sepsis-induced mortality by repressing hypotension induced renal circulatory dysfunction. Further, ABCF1 is necessary to maintain macrophage polarization in M2b state and the lack of ABCF1 shifts the state to the pro-inflammatory M1 state 53.

The molecular details of the ABCF1 switch are as follows. In the MyD88 pathway (M1 macrophage-like), the early phase of TLR4 signalling leads to UBC13 targeting TRAF6 for K63-polyubiquitination, which further targets cIAP1/2 for K63-polyubiquitination. cIAP1/2 then enhances K48-proteasomal degradation of ABCF1 and TRAF3. In the absence of ABCF1, TAK1 is phosphorylated, which leads to activation of MAPK and NF-kB pathways and elevated production of pro-inflammatory cytokines like TNFα, IL-1b, IL-6, thereby polarizing macrophages to M1 phenotype. Subsequently in the TRIF pathway (M2 macrophage-like), self K48-proteasomal degradation of cIAP1/2 results in K63-polyubiquitination of ABCF1 by TRAF6, which results in ABCF1 to bind and forms a complex with TRAF3 and SYK leading to the formation of K63-polyubiquitylated TRAF3 and SYK. This leads to TLR4 endocytosis into the endosomes, which then initiates TRIF-dependent TLR4 signalling and eventual production of IFN-I stimulated genes. This triggers phosphorylation of TBK1 that leads to phosphorylation and eventual dimerization of IRF3 and production of IFN-I stimulated genes. This shift from MyD88 to TRIF signalling by ABCF1 leads to increased production of IL-10, minimal production of TNFα, IL-1b, IL-6 and CD86, MHC-1l surface markers and decreased CD206 levels, thus polarizing macrophages to M2b phenotype.

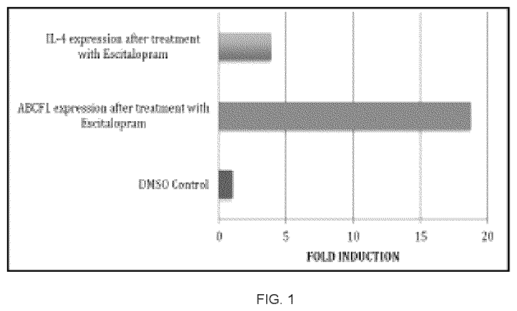

Escitalopram is a selective serotonin reuptake inhibitor (SSRI) and has the highest selectivity for the serotonin transporter compared to the norepinephrine transporter, making the side-effect profile relatively mild in comparison to less selective SSRIs⁵⁴. Additionally, noradrenergic or serotonin-norepinephrine reuptake inhibitors used to treat major depressive disorder have anti-inflammatory properties in vitro⁵⁵. Antidepressants, such as escitalopram, appear to possess anti-inflammatory properties^{56,57,58-60}. Mechanistically, antidepressants likely mediate this through a reduction in pro-inflammatory cytokines IL-1b, TNFα, and IL-6 with a reciprocal increase in anti-inflammatory cytokines including IL-10α. Studies have also shown that single nucleotide polymorphisms in IL-6 and IL-11, and mRNA levels of TNFα, are predictive of clinical response to the SSRI, escitalopram^{63,64}. Also, escitalopram modulates mRNA levels of cytokines in mouse brain⁶⁵ and decreases cytokine mRNA levels in the circulating immune cells of depression patients⁶⁰. Furthermore, IL-6 mRNA levels correlate to clinical response in depressed patients treated with antidepressants⁶⁰, and several cytokines, including IL-1b and TNFα, acutely stimulate serotonin transporter activity in neurons. The alteration of transport activity in serotonergic neurons in the brain provides a mechanism by which cytokines can modulate serotonergic signaling, and subsequently influence emotional cognitive processing. Experimental induction in humans with immune activators, such as LPS that activate macrophages and microglia, act as key drivers of depression and reveal that the severity of depressive symptoms is correlated with elevated blood levels of pro-inflammatory cytokines 33-35. Recently, ABCF1 was identified as a putative therapeutic target of escitalopram⁶⁶. In conjunction with the Genome-Based Therapeutic Drugs for Depression Project, published with the title “ABCF1 is identified as a putative therapeutic target of escitalopram in the inflammatory cytokine pathway,” the authors found that the peripheral blood mononuclear cells (PBMCs) of patients responding to escitalopram treatment subsequently increased the transcription of a single gene: ABCF1⁶⁶. Therefore, the effectiveness of a commonly used selective serotonin reuptake inhibitor correlates with ABCF1 expression. Furthermore, to confirm the observation that ABCF1 is elevated in the PBMCs of MDD patients is observable in vitro as well it was found that escitalopram induces IL-4 by approximately 5 fold and ABCF1 by approximately 20-fold in the macrophage cell line consistent with polarization towards an M2 phenotype (FIG. 1). Thus, ABCF1 may be a therapeutic target as it appears to reside at the intersection between inflammatory diseases and psychiatric illness.

### Example 2: The Effect of Psylocibin, Psylocin and their Analogs on ABCF1 Transcription

Preparation of Cells:
1. Macrophage cell line RAW264.7 (ATCC) were grown to 80% confluency in growth media (DMEM+10% FBS+glutamine).
2. Dilutions of the drugs were made at desired final concentrations for a Dose response experiment. The concentrations' used for this experiment are: 10 nM, 100 nM, 500 nM for Psilocin, Psylocibin, 4-Acetoxy-N, N-dimthyltryptamine, O-Acetyl Psilocin Fumerate, and 4-acetoxyindole.
3. A time course response experiment was done for the above stated drugs at concentrations mentioned above (10 nM, 100 nM, 500 nM). The time points chosen were 0, 30 mins, 2 hours and 24 hour.
4. Untreated cells were used as negative control and Escitalopram at 0.3 mM was used as a positive control to activate ABCF1 expression for all the experiments.

Analysis by qPCR:

Primers Used:

GAPDH FP:
TGGATTTGGACGCATTGGTC
GAPDH RP:
TTTGCACTGGTACGTGTTGAT
ABCF1 FP:
AGAAAGCCCGAGTTGTGTTTG
ABCF1 RP:
GCCCCCTTGTAGTCGTTGATG

1. Post treatment with drugs at different time points, the reaction was stopped by removing the media with the drug. Cells were then collected and RNA was isolated from these.
2. After checking the quality of the RNA, cDNA was generated and qPCR was run with ABCF1 primers as the target gene and GAPDH as the house keeping gene.
3. Normalized against the expression level of GAPDH, the fold change expression level of ABCF1 was calculated and tabulated for all treatment conditions.

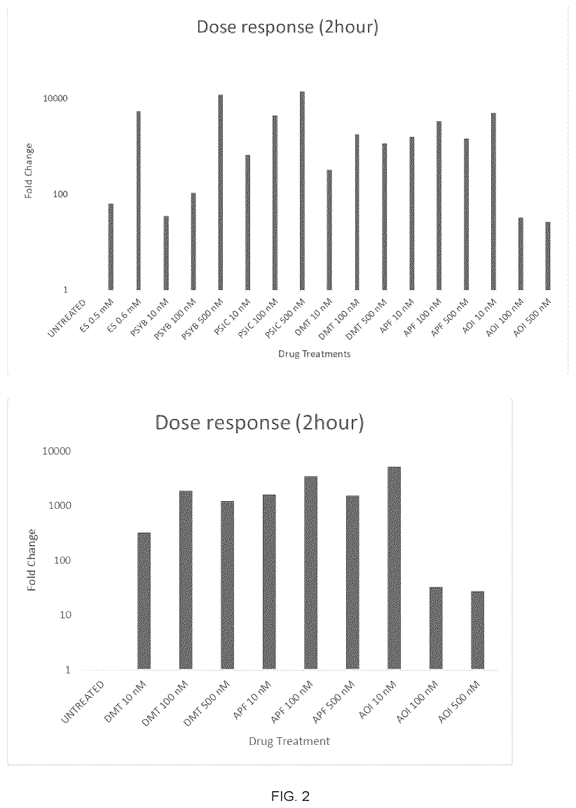

The results as set forth in FIG. 2 show psylocibin, psylocin and their analogs upregulate ABCF1 transcription.

### REFERENCES

1. Frohlich, F. Network neuroscience. (Elsevier/Academic Press, 2016).
2. Gotlib I H, H. C. Handbook of Depression. (The Guildford Press, 2009).
3. World_Health_Organization. Depression Fact Sheet, <https://www.who.int/news-room/factsheets/detail/depression> (2020).
4. Malhi, G. S. & Mann, J. J. Lancet 392, 2299-2312, doi:10.1016/S0140-6736(18)31948-2 (2018).
5. Herrman, H. et al. Lancet, doi:10.1016/S0140-6736(18)32408-5 (2018).
6. Cepeda, M. S., et al, Journal of Clinical Psychiatry 77, 1666-1671,
7. Zunszain, et al. in Behavioral Neurobiology of Depression and Its Treatment. Current Topics in Behavioral Neurosciences Vol. 14 Current Topics in Behavioral Neurosciences (eds P. Cowen, T. Sharp, & J. Lau) 135-151 (Springer, 2012).
8. Hasler, G. World Psychiatry: Official Journal of the World Psychiatry Association 9, 155-161 (2013).
9. Dean, J. & Keshavan, M. Asian J Psychiatr 27, 101-111 (2017).
10. Lederberg, J. & McCray, A. T. Scientist 15, 8-8 (2001).
11. Maes, M., et al. Neuroendocrinol Lett 29, 117-124 (2008).
12. Maes, M., et al. J Affect Disorders 141, 55-62, (2012).
13. Maes, M. et al. Acta Psychiat Scand 127, 344-354,
(2013).
14. Clarke, G. et al. Mol Psychiatry 18, 666-673, (2013).
15. Cheung, S. G. et al. Front Psychiatry 10, 34, (2019).
16. Valles-Colomer, M. et al. Nat Microbiol 4, 623-632, (2019).
17. Ellul, P., et al, D. Front Neurol 9, 167, (2018).
18. Chen, Y. et al. Psychiatry Res 188, 224-230, (2011).
19. Saito, S et al. Am J Reprod Immunol 63, 601-610 (2010).
20. Meltzer-Brody, S. Dialogues Clin Neurosci 13, 89-100 (2011).
21. Mastorakos, G. & Ilias, I. Ann N YAcad Sci 997, 136-149, (2003).
22. Zhang, L., Zhang, J. & You, Z. Front Cell Neurosci 12, 306, (2018).
23. Rosenblat, J. D., et al Prog Neuropsychopharmacol Biol Psychiatry 53, 23-34, doi:10.1016/j.pnpbp.2014.01.013 (2014).
24. Wohleb, E. S. et al; Psychoneuroendocrinology 37, 1491-1505, (2012).
25. Wohleb, E. S. et al. J Neurosci 31, 6277-6288, (2011).
26 Czeh, M., Gressens, P. & Kaindl, A. M. Dev Neurosci 33, 199-209, (2011).
27 Venneti, S., Lopresti, B. J. & Wiley, C. A. Glia 61, 10-23, (2013).
28 Yirmiya, R. et al. Adv Exp Med Biol 461, 283-316, (1999).
29 Rock, R. B. et al. Clin Microbiol Rev 17, 942-964, (2004).
30 Vollmer-Conna, U. et al. Psychol Med 34, 1289-1297, (2004).
31 Dowlati, Y. et al. Biol Psychiatry 67, 446-457, (2010).
32 Haapakoski, R., et al. Brain Behav Immun 49, 206-215, (2015).
33. Reichenberg, A. et al. Arch Gen Psychiatry 58, 445-452, (2001).
34. Grigoleit, J. S. et al. PLoS One 6, e28330, doi:10.1371/journal.pone.0028330 (2011).
35. Harrison, N. A. et al. Biol Psychiatry 66, 407-414, (2009).
36. Yirmiya, R. Brain Res 711, 163-174, (1996).
37. Yirmiya, R. et al. Neuropsychopharmacol 24, 531-544,(2001).
38. Henry, C. J. et al. J Neuroinflammation 5, 15, (2008).
39. O'Connor, J. C. et al. Mol Psychiatry 14, 511-522, (2009).
40. O'Connor, J. C. et al. J Neurosci 29, 4200-4209, (2009).
41. Dobos, N. et al. J Alzheimers Dis 28, 905-915, (2012).
42. Fenn, A. M. et al. Biol Psychiatry 76, 575-584, (2014).
43. Corona, A. W. et al. Brain Behav Immun 31, 134-142, (2013).
44. Li, Z. Q., et al. Biochem Biophys Res Commun 501, 933-940, (2018).
45. Garcia-Barrio, M., et al. EMBO J 19, 1887-1899, (2000).
46. Marton, M. J., et al. Mol Cell Biol 17, 4474-4489 (1997).
47. Paytubi, S., et al. Biochem J 409, 223-231, (2008).
48. Campbell, S. G., et al. J Cell Biol 170, 925-934, (2005).
49. Pestova, T. V. & Hellen, C. U. Nat Struct Mol Biol 13, 98-99, (2006).
50. Richard, M., et al. Genomics 53, 137-145, (1998).
51. Ota, M. et al. Immunogenetics 59, 45-52, (2007).
52. Zhu, H. et al. PLoS One 11, e0167212, (2016).
53. Arora, H. et al. Immunity 50, 418-431 e416, (2019).
54. Brunton, L., et al The Pharmacological Basis of Therapeutics. 12 edn, (McGraw Hill Professional, 2010).
55. Horowitz, M. A. et al. Int J Neuropsychopharmacol 18, (2014).
56. Abdel-Salam, O. M., et al Pharmacol Res 48, 157-165 (2003).
57. Roumestan, C. et al. Respir Res 8, 35, (2007).
58. Tynan, R. J. et al. Brain Behav Immun 26, 469-479, (2012).
59. AI-Amin, M. M., et al. Inflammopharmacology 21, 421-425, (2013).
60. Cattaneo, A. et al. Neuropsychopharmacology 38, 377-385, (2013).
61. Kenis, G. & Maes, M. Int J Neuropsychopharmacol 5, 401-412, (2002).
62. Kubera, M. et al. J Clin Psychopharmacol 21, 199-206 (2001).
63. Powell, T. R. et al. Eur Neuropsychopharmacol 23, 1105-1114, (2013).
64. Uher, R. et al. Am J Psychiatry 167, 555-564, (2010).
65. Malki, K. et al. Pharmacogenet Genomics 22, 765-776, (2012).
66. Powell, T. R. et al. J Psychopharmacol 27, 609-615, (2013).
67. Wilcox, S. M. et al. Plos One 12, e0175918, (2017).
68. Kimura-Ohba, S. et al. J Cereb Blood Flow Metab 36, 1731-1743, (2016).
69. Albert, P. R. J Psychiatry Neurosci 40, 219-221 (2015).
70. Yurist-Doutsch, S. et al. Plos One 11, e0159676, (2016).
71. Bartram, A. K et al. Appl Environ Microbiol 77, 3846-3852, (2011).
72. Schloss, P. D. et al. Appl Environ Microbiol 75, 7537-7541, (2009).
73. Hartmann, M. et al. ISME J 6, 2199-2218, (2012).
74. DeSantis, T. Z. et al. Appl Environ Microbiol 72, 5069-5072, (2006).
75. McMurdie, P. J. & Holmes, S. Plos One 8, e61217, (2013).
76. Christoffel, D. J. et al. J Neurosci 31, 314-321, (2011).
77. Coll, R. C. et al. Nat Med 21, 248-255, (2015).
78. Golden, S. A., et al Nat Protoc 6, 1183-1191, (2011).
79 McGirr, A., et al. Brain 140, 2210-2225, (2017).
80 Brady, A. M. & Floresco, S. B. Vis Exp, e52387 (2015).
81 Weiss, J. M., et al J Comp Physiol Psychol 72, 153-160 (1970).
82 Wagner, H. R., 2nd, Hall, T. L. & Cote, I. L. J Gen Psychol 96, 313-318, (1977).
83 Porsolt, R. D., et al. Arch Int Pharmacodyn Ther 229, 327-336 (1977).
84 Steru, L., et al. Psychopharmacology (Berl) 85, 367-370 (1985).
85 Liu, M. Y. et al. Nat Protoc 13, 1686-1698, (2018).
86 Sunyer, B., et al. Barnes maze, a useful task to assess spatial reference memory in the mice (2007).

1. A method of inhibiting neuroinflammation in a patient in need thereof, said method comprising administering an agonist of ABCF1.

2. A method inhibiting neuroinflammation in a patient in need thereof, said method comprising administering an ABCF1 protein or a polynucleotide encoding ABCF1.

3. The method of claim 1 or 2 wherein inhibition of neuroinflammation treats or alleviates one or more symptoms of depression in said patient.

4. The method of claim 3, wherein the one or more symptoms are selected from the group consisting of trouble concentrating, remembering details, and making decisions; fatigue; feelings of guilt, worthlessness, and helplessness; pessimism and hopelessness; insomnia, early-morning wakefulness, or sleeping too much; irritability; restlessness; loss of interest in things once pleasurable, including sex; overeating, or appetite loss; aches, pains, headaches, or cramps that won't go away; digestive problems that don't get better, even with treatment; persistent sad, anxious, or “empty” feelings; suicidal thoughts or attempts and combinations thereof.

5. The method of any one of claims 1 to 4, wherein the patient is a patient with an autoimmune disease.

6. The method of claim 5, wherein the autoimmune disease is inflammatory bowel disease, rheumatoid arthritis, or pancreatitis.

7. The method of claim 6, wherein the inflammatory bowel disease is Crohn's disease or ulcerative colitis.

8. The method of any one of claims 5 to 7, wherein said agonist treats or alleviates one or more symptoms of said autoimmune disease.

9. A method of preventing and/or treating Major Depressive Disorder, said method comprising administering an agonist of ABCF1.

10. A method of preventing and/or treating Major Depressive Disorder, said method comprising administering an ABCF1 protein or a polynucleotide encoding ABCF1.

11. A method of treating an autoimmune disease and comorbid neuropsychiatric disorders, said method comprising administering an agonist of ABCF1.

12. A method of treating an autoimmune disease and comorbid neuropsychiatric disorders, said method comprising administering an ABCF1 protein or a polynucleotide encoding ABCF1.

13. A method of preventing and/or treating a depressive disorder associated with inflammation, said method comprising administering an agonist of ABCF1.

14. The method of any one of claims 1, 3 to 9, 11 and 13, wherein the agonist is psylocibin, psylocin or an analog or derivative thereof.

15. The method of any one of claims 1, 3 to 9, 11 and 13, wherein the agonist is Psilocin, Psylocibin, 4-Acetoxy-N, N-dimthyltryptamine, O-Acetyl Psilocin Fumerate, and 4-acetoxyindole.

16. The method of any one of claims 1, 3 to 9, 11 and 13, wherein the agonist is a compound set forth in Table 1 or 2.

17. The method of any one of claims 1, 3 to 9, 11 and 13, further comprising administration of another therapeutic agent.

18. The method of claim 13, wherein the inflammation is neuroinflammation.
